(19) United States
(12) Patent Application Publication
(10) Pub. No.: US 2016/0066565 A1
(43) Pub. Date: Mar. 10, 2016

(54) ISOLATOR DEVICES FOR COLLECTION, PARAMETRICAL CHARACTERIZATION AND LONG-TERM PRESERVATION OF ORGANIC FLUIDS AND/OR MATERIALS CONTAINING STEM CELLS

(71) Applicant: BIOCELL CENTER CORPORATION, Medford, MA (US)

(72) Inventors: MARCO GIOVANNI REGUZZONI, BUSTO ARSIZIO (IT); SIMONE BERTONI, CASTRONNO (IT)

(21) Appl. No.: 14/934,791

(22) Filed: Nov. 6, 2015

Related U.S. Application Data
(63) Continuation-in-part of application No. 12/747,437, filed on Sep. 10, 2010, now abandoned.

(51) Int. Cl. A01N 1/02; G01N 33/50

(52) U.S. Cl. CPC A01N 1/0252; G01N 33/5005

(57) ABSTRACT

An improved isolator for collection, parametrical characterization and long-term preservation of samples containing stem cells comprises: a work chamber (2) isolated from the external environment; gloves jutting out at an inside of said work chamber (2); an inlet chamber (4) for introduction of a sample to be processed; an outlet chamber connected with the work chamber (2) and having a sterile sample-collecting bag; sterilizing means acting on said sample and/or on said inside of the work chamber (2); filtering means; cooling means for bringing and keeping said sample at a constant working temperature throughout the inlet chamber (4), the work chamber (2) and the outlet chamber; and visual imaging means (5) focused on the sample and defining a visual path which maintains a complete mechanical and pneumatic/hydraulic isolation between said inside of the work chamber (2) and the external environment.

### FIELD OF THE INVENTION

The present invention relates to various improvements in so-called “isolation devices” employable in methods of quantitative/parametric characterization, collection and long-term preservation of fluids and/or materials containing stem cells (such as, for example, amniotic fluid, chorionic villi and/or placenta).

### DESCRIPTION OF RELATED ART

It is known that throughout the various stages of a woman's pregnancy a wide range of clinical examinations are made: such examinations often rely on taking from the woman herself very small samples of organic material which then undergo a series of capable laboratory essays in order to determinate the foetus' development conditions and the health state of both mother and foetus themselves.

In the clinical field tests such as amniocentesis and CVS (i.e. Chorionic Villus Sampling) are currently being executed, and at birth the expelled placenta can also be sampled for clinical use.

Due to the high criticalities related to the execution of amniocentesis (and, in a less hampering way, to the execution of CVS as well), a very limited amount of organic matter is taken from the mother's body: as a rule of thumb, amniotic liquid samples range in volume from about 3 cm³ in volume up to no more than 10 cm³.

Moreover, after being extracted from the mother's body, a large share of the amniotic fluid is devoted to clinical tests, and therefore a very limited quantity of this fluid is made available for purposes of stem cells' collection and future growth/multiplication.

It is now to be pointed out that presently the known art does not contemplate any method involving long-term use of the amniotic liquid or chorionic villi, above all in connection with the possibility of preserving, taking out and subsequently manipulating the extremely low numbers of stem cells that can be found in the very small volumes/masses of organic material sampled during amniocentesis or CVS . . . nor there is a known methodology for maximizing the “efficiency” (in terms of maximization of the chance of retaining viable stem cells after a long storage period) of sampling, quantitatively characterizing and storing a sample of human placenta (or at least a portion of chorionic villi extracted or separated by a sample of human placenta) to be used for stem cells cultivation and multiplication.

In terms of clinical protocols related to storing the so-called “autologous” stem cells, a huge drawback is therefore given by the very small count of stem cells primarily available from the sampling acts: this small “initial” number leads to even more relevant difficulties of growing-up a population of stem cells starting from the sample, even more remarkably after a long period of storage of the sample itself.

In terms of drawbacks, it is therefore known that execution of known methods for sampling of fluids and/or materials (of organic or non-organic nature) involves statistically high risks of, contamination of the taken samples, with all negative consequences resulting therefrom and with an even more damaging effect consisting in further reduction of the number of stem cells effectively kept in viable/multipliable conditions: such risks can be limited by taking a much more significant “initial volume” of the organic sample, but this is not always possible (e.g. in the amniocentesis).

In addition, referring in particular to organic fluids and/or materials containing stem cells, it is to be pointed out that implementation of a method enabling an efficient and accurate collection and preservation of same is hitherto unknown, neither known is a method capable of ensuring the optimal conditions for putting into practice the “working” and manipulation techniques that can (or could in the future) be applied to the stem cells themselves in order to have the maximum degree of growth and multiplication starting from a very low number of pre-selected and parametrically characterized stem cells in an organic sample.

In the light of the above, the present invention aims at conceiving an improved “isolator” device which is able to obviate the set out drawbacks, in particular with reference to the limitation of the “post-storage” viability and availability of stem cells, and even in terms of enhancement of the working conditions of specialized operators that work on the sampled organic material in order to obtain its parametric characterization and in order to prepare the sample itself for a long-term storage.

Referring in particular to working conditions of the organic sample, the present invention aims also to ensure perfect and time-constant sterility and physio-chemical stability of the sample itself at least within all the working areas of the isolator device where the parametric characterization and preparation-for-storage operations occur.

It is also an aim of the present invention to provide an “isolator” device having high structural efficiency, widespread functionality, great ease of sterilization and maintenance, high-precision manipulation capability of the collected fluid and/or organic material and low production costs.

From the standpoint the working method (or otherwise stated, in terms of laboratory protocols), the present invention aims at conceiving a sampling, parametric characterization, preparation-for-storage and long-term storage overall process enabling the attainment of high reliability rates on the samples themselves, in terms of capability of growth and multiplication of the stem cells therein contained and in terms of speed and accuracy of the working steps: still more generally, the present invention aims at conceiving a method capable of sampling and preserving organic fluids or materials (amniotic fluid and/or chorionic villi and/or placenta or, according to the various operative requirements even other kinds of organic samples such as amniotic membrane and/or umbilical cord and so on) containing stem cells, so that it is possible to work on the stem cells therein contained and maintained in a preservation state, even after an indefinite period of storage.

Still as regards the method, the present invention wishes to make available a process enabling possible admixing of preserving substances to the sampled fluid and/or material, in a short period of time and with the greatest reliability and accuracy and with the least obnoxious (e.g.: mutagenic and/or teratogenic or more in general cytotoxic) effects on the stem cells, in particular during the step of adding preservative substances to the organic samples (it is known that preserving agents may have a strong killing rate on stem cells if they are not infused into the samples within very narrow ranges).

### SUMMARY OF THE INVENTION

The foregoing and further aims are achieved by improvements in isolator devices for collection, parametrical characterization and long-term preservation of organic fluids and/or materials containing stem cells according to the invention (and by a laboratory protocol or method employing such an improved isolator device) in accordance with the present invention, having the features shown in the appended claims and hereinafter illustrated in an embodiment thereof given by way of non-limiting example, as well as in the accompanying drawings.

### BRIEF DESCRIPTION OF THE DRAWINGS

FIG. 1 is a diagrammatic view of a first embodiment of a device in accordance with the invention; and

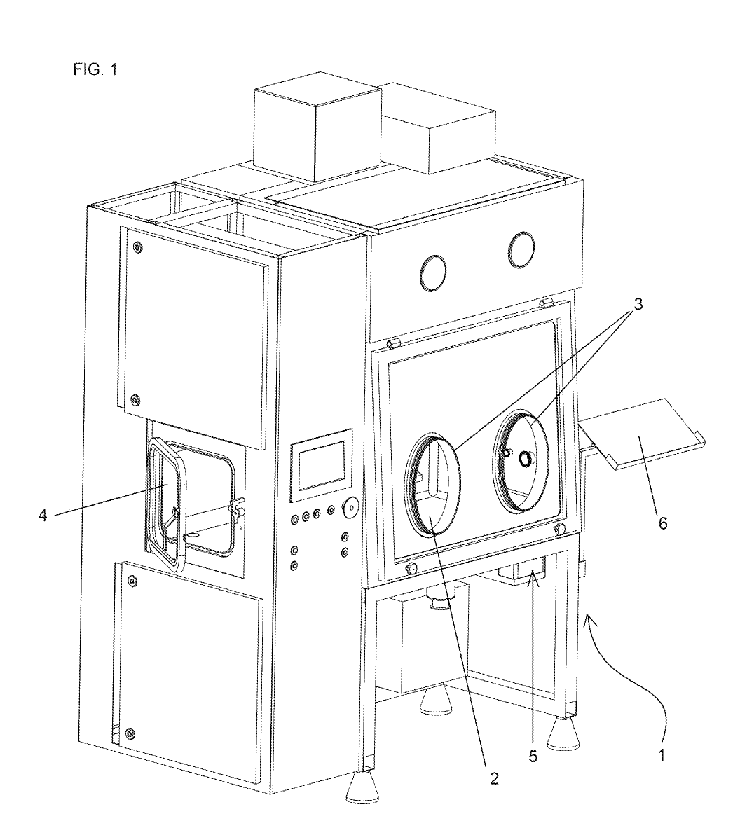

FIG. 2 is an enhanced view of the part evidenced as “section I-I” in FIG. 1.

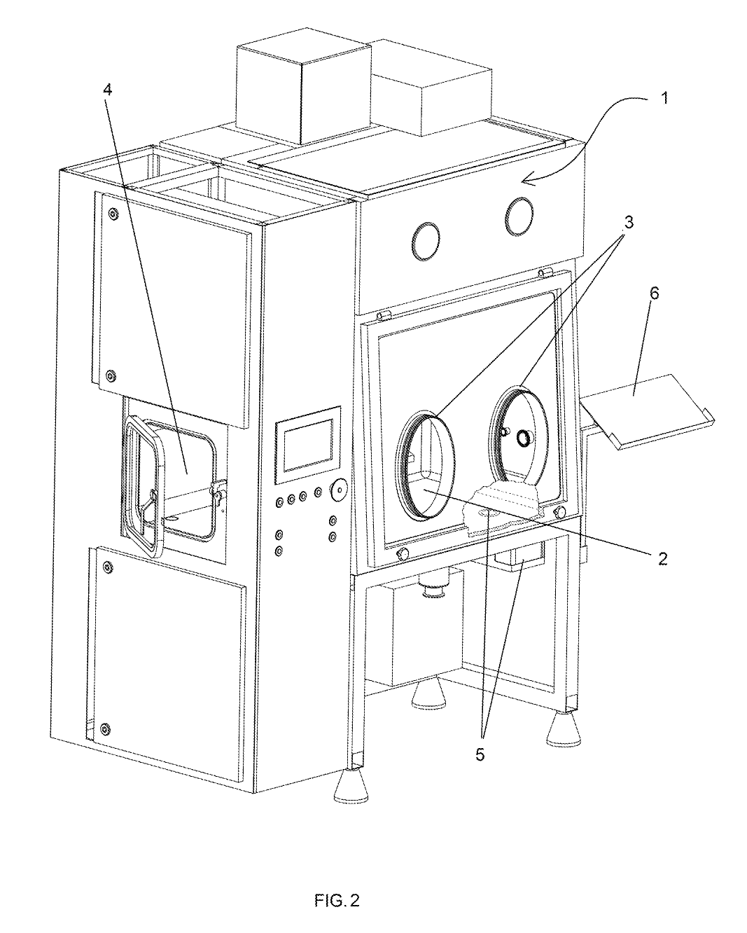

### DETAILED DESCRIPTION OF PREFERRED EMBODIMENTS OF THE INVENTION

With reference to the drawings, the improved isolator according to the invention is mainly (but not exclusively) employed for collection, parametrical characterization and long-term preservation of organic fluids and/or materials containing stem cells and from a structural standpoint it mainly comprises a work chamber (2) isolated from the external environment: conveniently, this work chamber (2) is pressurized to ensure that no “external” atmosphere can enter in the work chamber (2) itself.

It should also be appreciated that the size of the isolator's work chamber (2) (which can be considered by way of example as a cubic volume having sides of about 80 cm) has been suitably studied so as to reduce the sizes of the inner surfaces and the overall volume: this geometric effect greatly reduces the time required for decontamination of the work chamber (2) and also reduces consumption of sterilizing agents required for decontamination.

In order to allow an operator to perform the suitable operations on the sample, gloves jutting out at an inside of said work chamber (2) are provided: the gloves are connected and sealed with one of the work chamber (2) walls in correspondence of their respective seating (3), and an operator is able to accede to the work chamber (2) by use of said gloves.

The isolator also comprise an inlet chamber (4) for introduction of a sample to be worked on (such a sample comprises in turn fluids and/or materials containing stem cells to be processed): the inlet chamber (4) is connected to the work chamber (2) and is provided with interlocking doors that do not allow direct communication between the work chamber (2) and the external environment.

As already said before, the work chamber (2) being pressurized to a greater pressure than the inlet chamber (4), in order to avoid atmospheric contamination.

The isolator may also comprise an outlet chamber, which can be coincident with the inlet chamber (4) or not, depending on the requirements, connected with the work chamber (2) and having a sterile sample-collecting bag: the outlet chamber being brought into communication with the work chamber (2) by means of interlocking doors.

At the same time the isolator is provided with sealed and/or hermetic and/or sterile packaging means located at the outlet chamber, so as to allow a packaging of the “product” (or otherwise stated, of the worked-on sample) coming out of the isolator in an aseptic manner.

An example of implementation of such hermetic and/or sterile packaging means is given by a so-called sterile “rolled bag”, which known in the art and which is useful for the purpose of carrying the sample to subsequent cryogenic freezing/preservation as it will be detailed later.

The outlet chamber of the isolator may also comprise suitable selecting means for allowing a separate extraction of said sample and of waste materials deriving from operations performed on the sample inside the work chamber (2): with reference to the annexed figures, these selecting means comprise a pass-box defining a dedicated waste material exit pathway through a bottom wall of the outlet chamber.

The presence of the just cited pass-box in addition to the presence of the sterile “rolled bag” or equivalent means enables elimination of eventual working waste without contaminating any other part of the isolator (and therefore avoiding supplementary sterilization cycles being carried out).

The isolator also comprises sterilizing means acting at least on the sample and/or on the inside of the work chamber (2): such sterilizing means are designed for being usable before a single working cycle (i.e.: a cycle wherein a single sample is introduced in the isolator and is undergoing to collection, parametric characterization and conservation steps) of the isolator and/or between working cycles relating to subsequent samples introduced in the isolator one at a time.

According to various possible embodiments of the invention, the sterilization means may be acting on the work chamber (2) and/or on the inlet chamber (4) and/or on the outlet chamber: in general the sterilizing means atomize one or more sterilizing agents within the isolator (e.g.: hydrogen peroxide H₂O₂) so as to decontaminate every point of the isolator itself.

Quantitatively speaking, the sterilizing means in the present invention are capable of introducing hydrogen peroxide (H₂O₂) at a temperature ranging from 90° C. to 110° C. over a period of time ranging from 30 seconds to 7 minutes: in this way an optimum balance between reduced operative times and satisfactory degree of sterilization of the various “insides” of the isolator is achieved.

It is important to observe that the entire cycle of sterilization of the internal volume of the isolator, considering the hydrogen peroxide introduction and its reaction time with the surfaces and with the internal atmosphere, ranges in time from 15 minutes to 3 hours.

The isolator also comprises suitable filtering means acting on an atmosphere of the inside of the work chamber (2) (and eventually, also on an inside of the inlet chamber (4) and/or an inside of the outlet chamber): such filtering means may comprise one or more “hepa” type filters and/or filters of other kind, made of gore-tex for example, that are operatively active on an admission line of the sterilizing means (e.g.: a line for admission of hydrogen peroxide H₂O₂); in this manner a further lowering of the decontamination time is obtained and a reduction of the overall working time required for a single samples' working cycle.

The isolator also comprises cooling means for bringing and keeping said sample at a constant working temperature throughout the inlet chamber (4), the work chamber (2) and the outlet chamber: said cooling means are capable of bringing and keeping the sample at a working temperature ranging from 2.5° C. to 5.5° C. within a transitory cooling period ranging from 5 minutes seconds to 15 minutes.

As regards the effects of the invention's cooling means on a single work cycle, it is to be remarked that the cooling means hereby provided are capable of keeping a sample within the aforecited working temperature for a period of time ranging from 40 minutes to 60 minutes throughout the inlet chamber (4), the work chamber (2) and the outlet chamber (so ensuring the attaining and maintaining of one critical parameter with direct influence on high survivability and residual viability of the stem cells in the sample).

Talking about temperature control and regulation, it can be seen that the isolator also offers the possibility of pre-cooling a suitable freezing solution (in which the sample will be dipped into after the operator has finished to perform the required collection and parametric characterization operations) by providing a thermo-block (usually made of steel) housed inside the work chamber (2): this positioning of the thermo-block ensures maximum sterility for both the sample and the inside working area, unlike known systems which exploit a more traditional cooling by ice (which is not sterile and cannot be sterilized) contained in a non-sterile container.

The structural features relating to temperature control & regulation in the isolator therefore allow keeping the sample inside the work chamber (2) at a controlled temperature so that capable “comfort conditions” can be achieved both for the product and the operator.

Advantageously, the isolator is provided with visual imaging means (5) focused on the sample (at least when the sample is contained or worked-on within the work chamber (2)): such visual imaging means (5) define a visual path which maintains a complete mechanical and pneumatic/hydraulic isolation between the inside space of the work chamber (2) and the external environment.

More in detail, the visual imaging means (5) comprise:
a camera capable of image and/or video caption focused on the sample within the work chamber (2);
an optically transparent window integrally inserted and flush-formed in a bottom wall of the work chamber (2) and allowing said visual path to be defined upwards from said camera and said sample; and
hermetic seals interposed between said optically transparent window and a locating cavity realized in said bottom wall of the work chamber (2) wherein said optically transparent window is mounted.

The structural features just described allow to caption a very highly detailed imagery of the sample during all the working operations performed by an operator: this is due to the fact that the operator moves his hands in the gloves within the inside space of the work chamber (2) and “above” the sample, while the visual path (and therefore the signal link between the camera and the sample) occupies a space “below” the sample.

At the same time, this general principle can be applied in other embodiments of the invention, where the visual imaging means (5) are structurally connected to another wall and where the visual path defined between the camera and the sample—and through that wall—does not come into interference with the operator's hands (or with the gloves jutting at the inside of the work chamber (2)).

In order to minimize problems related to small gaps (which are difficult to be reached by the sterilizing means and which therefore increase risks of work chamber (2)'s contamination), the camera is located outside the work chamber (2) and has a digital resolution ranging from 1 megapixel to 20 megapixels and a zoom factor ranging from 10× to 30× (for example, a zoom factor of 20×).

Minimization of critical areas/volumes which can be sources of bacterial growth or “nests” for obnoxious substances is also the driving principle for the choice to realize the mounting of the optically transparent window integrally inserted and flush-formed in the bottom wall: by minimizing/nullifying geometric discontinuities, such critical areas/volumes are equally minimized/nullified.

In order to give proper structural coherence and correct visual alignment, the isolator further comprises an attachment hub located adjacent to the optically transparent window (and of course located along the visual path): such an attachment hub bears the camera and conveniently comprises illuminating means, which in turn are aligned with the visual path and convey a flow of light towards the sample.

As an alternative, the illuminating means may be positioned anywhere else within the isolator's working space, provided that they guarantee an adequate alignment of their emitted light with respect to the optical axis of the visual path: for example, the illuminating means may comprise a light emitter positioned above the sample working area and protruding from a lateral wall of the isolator itself (in this case, electrical energy to this emitter is provided via suitable connections passing through the isolator's wall and adequately sealed for maintaining sterile conditions in the working chamber of the isolator itself).

In another embodiment of the present invention, the illuminating means comprise a crown of LEDs surrounding a lens of the camera in proximity of said attachment hub: remarkably, in order to maximize compactness of the device, the attachment hub having a diameter ranging from 1 cm to 3 cm so that highly compact cameras can be installed.

Whenever required by particular requirement of encumbrance or reduced space, the visual imaging means (5) further comprise optical devices aligned with the visual path to enhance and/or deflect and/or concentrate light and/or visual signals travelling along the visual path itself (e.g.: mirrors, lenses, prisms and devices for deflecting/reflecting/refracting light).

Beside this, the visual imaging means (5) may also comprise a TV display—or an analogous electronic equipment, such a tablet or a monitor connected with a PC—on where the captioned image of the sample (taken by the camera) is projected: more in general it can be seen that the isolator (1) further comprises electronic visualization, elaboration, correlation and memory storage means connected at least to the visual imaging means (5) (such electronic means may be integrated in a personal “laptop-type” computer residing in a specific shelf (6) integrally extending from the isolator (1), so that easy and short-range connection with other electronic/optical equipment of the isolator is rendered available).

The just cited electronic means are capable of performing visual image processing, storing and data correlating between a set of static images and/or videos taken on the sample and identification parameters associable to the sample itself: in order to have an unambiguous and complete description and characterization of each sample worked in the isolator, the identification parameters may comprise:
a particle count related to one or more chemical compounds within the sample;
a microbiological count related to one or more kinds of cells or organic entities, including stem cells, within the sample; and
information regarding a time of sampling, a provenance of sampling, including an identification of a patient/donor, and environmental conditions of sampling.

For operating purposes, the particle count and/or microbiological count parameters can be continuously picked up by choosing suitable sensors available on the market: this timely continuity of analysis ensures that during operator's work on the sample, the optimal conditions maximizing the survivability of the stem cells enclosed in the sample (and then the viability of the same stem cells after the sample has been “worked” in the isolator and stored).

For completion of the different operating aspects of the isolator, such suitable sensors act at least in the work chamber (2) (but, if necessary, also in the inlet chamber (4) and/or the outlet chamber) and can measure the following parameters:
a microbiological count (in this case the sensors can be placed in proximity of the sample, so as to have a truer and safer data perception);
an air speed within the work chamber (2) (in this case the sensors be coupled with the aforecited filtering means, or they can be mounted in suitable venting means acting on the work chamber (2) and/or the inlet chamber (4) and/or the outlet chamber);
a particle count related to presence/density of particles suspended within the inside space of the work chamber (2); and/or
a possible residual amount of sterilizing substance which may rest in the work chamber (2) after an engagement of the sterilizing means which have been described before.

For the complete treatment of samples in terms of ensuring their best conditions for storage and capability of growth of the stem cells' population, the isolator further comprises spraying and/or mixing means active on the sample for adding to it a preserving substance: in the illustrated embodiment, such spraying and/or mixing means are capable of adding a predetermined amount of dimethyl sulfoxide (DMSO) to the sample in an amount ranging from 9% to 11% with respect to a total volume of said sample.

Remarkably, the addition of dimethyl sulfoxide (DMSO) to the sample takes place at a constant working temperature ranging from 2.5° C. to 5.5° C., which is in turn ensured by the cooling means: this combination of DMSO percentage and temperature is crucial in avoiding obnoxious effects of the DMSO over the stem cells.

Regarding other useful (yet optional) features of the invention, the isolator further comprises positioning means for placing and/or moving the sample at least between a waiting position on the bottom wall of the work chamber (2) and an analysis-enabling position on the bottom wall of the work chamber (2): while the waiting position can be arbitrarily determined in the work chamber (2), the analysis-enabling position is usually univocally determined and is aligned with the visual path of the visual imaging means (5).

From a structural standpoint, the positioning means allow the normal functions of the visual imaging means (5), and at this scope they comprise:
a sample support having a see-through bottom portion resting inside the visual path in the analysis-enabling position;
an engagement projection connected to the sample support and protruding towards the bottom wall of the work chamber (2) (otherwise stated, the engagement projection rests outside the visual path in the analysis-enabling position of said sample support); and
a predetermined number (one or more, depending on the possible embodiments of the isolator) of engagement seats located in the bottom wall of the work chamber (2).

In order to allow a perfect mutual fit, the engagement seats are counter-shaped to the engagement projection of the sample support, and at the same time the engagement seat corresponding to the analysis-enabling position of the sample supports bears the optically transparent window described hereabove.

The positioning means further comprise releasable clamp locks to be selectively operated on the sample when it is placed in the sample support: these clamp locks improve stability and manipulability of the sample with regard to the operator's various actions.

The isolator further comprises stirring means active on the sample and/or on the preserving substance within the work chamber (2): such stirring means are remarkably connected to a mechanical energy source located outside the work chamber (2) through contactless transmission means, so as to avoid a possible entry pathway for contaminating agents within the work chamber (2) itself and so as to minimize maintenance issues or mechanical failures.

In the herein illustrated embodiment, the stirring means comprise a stirring body made of ferromagnetic material, and the contactless transmission means comprise a spatially rotatable/variable magnetic field acting on the stirring body through walls of the work chamber (2).

In order to reach an ergonomic improvement for the operator, the isolator also has positioning means for a biological sample (or in other words, for the sampled fluid and/or material), as well as for the material required for processing: conveniently, these means can consist of a steel rack having the same surface finish degree as the isolator's walls.

The isolator can also be provided with self-governing movement means (a train of pivoting wheels or the like, for example) that allow displacement of same inside the room: practically, the self-governing-movement means allows the isolator to be shifted to the desired points, thus facilitating cleaning of the room and maintenance of the isolator itself, for example (or in any case offering the possibility of shifting the isolator to points in the room or laboratory that are more advantageous from an operating point of view).

From a functional point of view, the improved isolator can be effectively employed in a method dedicated to sampling, parametric characterization and preparation for long-term storage of (organic) fluids and/or materials containing a certain amount of stem cells: such a method is particularly appreciable in the clinical field, where for instance it is possible to operate on the amniotic liquid or also, if necessary, on an organic sample comprising a predetermined amount of chorionic villi (or again, on a capable amount of placenta); this method can this be used both in post-extraction treatment of samples coming from traditional amniocentesis and/or CVS procedures.

The method of collection, parametrical characterization and long-term preservation of organic fluids and/or materials containing stem cells is implementable on fluids and/or materials comprising an amniotic liquid and/or chorionic villi and/or placenta, and comprises the following steps:
providing a sample of organic fluid and/or material containing stem cells that is closed and/or separated from the external environment, the sample having a total volume ranging from 1.8 cm³ to 10 cm³;
bringing the sample to a constant working temperature ranging from 2.5° C. to 5.5° C. within a transitory cooling period ranging from 5 minutes to 15 minutes;
adding a predetermined amount of a preserving substance ranging from 9% to 11% (e.g. 10%) with respect to the total volume of said sample, the sample being kept at said constant working temperature; and
keeping the sample added with said preserving substance at said constant working temperature for a period of time ranging from 40 min to 60 min throughout an inlet chamber (4), a work chamber (2) and an outlet chamber of said isolator.

Of course, during the execution of the main steps just presented, other intervention on the samples can be performed, but all of these interventions must be conducted in an overall time window and in environmental “internal” conditions of the isolator that allow a high rate of stem cells' survivability (at least 75% of the original stem cells content of the sample) and/or stem cells' viability.

As an example of further possible method steps, the following ones can be cited:
detecting a set of static images and/or videos taken on the sample;
identificating parameters associable to the sample itself, said identification parameters comprising:
a particle count related to one or more chemical compounds within the sample;
a microbiological count related to one or more kinds of cells or organic entities, including stem cells, within the sample; and
information regarding a time of sampling, a provenance of sampling (including an identification of a patient/donor) and environmental conditions of sampling; and
correlating and storing the set of static images and/or videos and/or the identificating parameters to the sample.

An extra set of possible “further operations and/or interventions” can also comprise the following (in particular, after having added the sample with the preserving substance and before delivering it to cryopreservation):
freezing the sample;
transferring the sample from the isolator into cryogenic storage means; and
allocating data related to the transferring step of the sample into cryogenic storage means with the set of static images and/or videos taken on the sample and with the identificating parameters associable to the sample itself.

From a clinical point of view, the step of freezing the sample may be carried out on non-hematic samples and on previously taken samples free of meconium, and the step of providing a sample may be performed through amniocentesis and/or a chorionic villus sampling procedure (CVS) and/or through selection and/or excision/seizing of a predetermined amount of placenta (along with a subsequent confinement of the sample into a collection volume).

Remarkably, the method according to this invention integrates a step of adding, e.g. by spraying and/or mixing, the sample to a preserving substance (for example a dimethyl sulfoxide-based preservative, or DMSO as commonly termed in the chemical field).

More in detail, it is possible to see that the adding sub-step is carried out through selective determination of the “moment” for mixing the preservative with the fluid and/or material and through selective determination of capable temperature conditions wherein the spraying/mixing step can take place with minimization/nullification of cytotoxic effects on the stem cells.

In accordance with the present invention, the adding step too takes place entirely within the improved isolator device, while ensuring the full separation and hermetic sealing of the collected fluid and/or material relative to the surrounding external environment.

Conveniently, the step of preserving the “added” sample comprises a sub-step of cooling the fluid and/or material under a predetermined preservation temperature; this cooling sub-step can precede the adding step.

After the adding step and after possibly doing all the necessary operations/interventions to enable preservation of the added sample for an indefinite period of time, it is also possible to transfer the sample into suitable storage means.

This step of transferring the fluid and/or material into storage means can be conveniently optimized from the logistic point of view by allocating a series of data at least concerning positioning (but also, depending on specific requirements, data relating to other parameters, such as sampling data, environmental conditions at the sampling moment, operator who has carried out sampling, and so on) to each sample; subsequently, these data can be stored into electronic storage systems.

In practical terms, the high survivability and “residual” viability (that is, post long-term storage viability) of the stem cells allow the integration of the aforecited method into laboratory protocol related to subsequent new processing or culturing of the stem cells themselves: it is to be noted in this connection that stem cells taken and “re-generated” from the mother's amniotic liquid and/or chorionic villi and/or placenta can be used as an autologous cellular therapy source for treatment of pathologies in humans (and so, in order to ensure use of these cells it is of the greatest importance to ensure the maximum level of availability, survivability and viability of the stem cells within the original “worked-on” sample).

Again, it is to be noted that samples coming from particular extraction procedures such as amniocentesis or CVS can be very small and therefore which can contain a very low number of stem cells: maximizing the chances of growing up a significant amount of perfectly compatible stem cells to be re-inoculated in a patient is therefore a highly praised goal made possible by preventive application of this invention to laboratory protocols.

It is to be noted at this point that use of the improved isolator constitutes an important novelty relative to the methodologies presently applied for manipulating stem cells: actually, operators presently acting on stem cells move (and manipulate) the biological material inside sterile rooms and therefore, although having all kinds of available protections (masks, gloves and others), they yet represent a contamination source for the organic fluid or material under processing since they transfer (at least through breathing) an important charge of bacteria or in any case of polluting agents (powders and dust, residues resulting from exfoliation of the skin and so on) into the same environment where the stem cells are.

On the contrary, use of the isolator enables full separation between the operator (and above all any environmental alteration/pollution source connected with the operator's physical presence) and the sample containing the stem cells.

In terms of integration between a typical laboratory protocol and the steps performed in accordance with the inventions, it can be noted that at least a sample coming from amniocentesis or CVS (or from extraction/excision of placenta) can be contained in a 15 cm³ test tube with conical bottom and screw plug: such tubes are then centrifuged at 2000 rpm for 10 minutes.

After centrifugation, the test tube is inserted into the isolator's main work chamber (2) and without disturbing the so-called “cellular pellet”, the so-called “supernatant” is taken and preserved for preparing the freezing solution for the sample with DMSO.

This solution is cooled inside the isolator and 1 ml thereof is used to suspend the amniocyte pellet again, said pellet being then inserted into another test tube for freezing, e.g. obtained by suitable programmable freezer means.

At the end of the freezing step, the sample is preserved in cryogenic storage containers containing liquid nitrogen: freezing can be conveniently carried out on non-hematic samples and on samples non containing meconium that have been taken 24-48 hours earlier.

Whenever needed, defrosting of the sample may be carried out by taking the sample out of the liquid nitrogen, positioning it in ice and bringing it into a 37° C. thermostat in the shortest period of time (e.g. about 3 minutes); then, the defrosted sample is transferred into the isolator and then again it is drop-wise transferred into a 15 cm³ test tube with conical bottom and screw plug (this test tube contains about 9 cm³ of washing medium).

At the end of the just described operation, the test tube is caused to come out of the isolator's outlet chamber and is subsequently centrifuged at 1500 rpm for 10 minutes: subsequently, the test tube is inserted into the isolator and without disturbing the cellular pellet, the supernatant is taken.

At this point, the cell-containing pellet is suspended again with a capable cell-growth substance (termed “medium”) in an amount of about 2 cm³; the cellular suspension will be transferred into a capable flask (in jargon termed “T25”) for expansion.

It is to be noted that a long-term (or even an undetermined time period) cryogenic preservation step and possibly a subsequent unstoring step that in turn comprises at least a defrosting sub-step can be accounted for.

As regards the above mentioned isolator, it is to be noted that within the scope of the present invention the structure of the latter has been suitably conceived for maximizing the efficiency of the overall work method, and for minimizing the working time on the samples when they are introduced in the isolator device for the first time.

Depending on different occurrences, the above described isolator can also be used separated from the method being the object of the present invention, i.e. in other industrial and/or laboratory processing methods on several different types of materials and/or (biological and non-biological) samples.

The invention enables achievement of important advantages both in terms of structural improvements of the isolator device and in terms of functional improvements to working protocols related to stem cells catalogation and preservation.

First of all, it is clear that usage of the present isolator device in the medical/clinical field allows higher availability, survivability and viability rates in the stem cells number/population, and also gains better hygiene and sterilization parameters to be maintained in the treated samples.

In terms of operation, the present invention therefore enables accomplishment of a quicker preliminary manipulation (comprehensive of parametric characterization and unambiguous identification of the samples) collection and preservation method as compared with traditional methods in use.

This method also allows two operations that are generally carried out at different times (and therefore are time-consuming) to be integrated into a single “time-shrinking” operating sequence, which is a key parameter for minimizing perturbations to the biological state of the stem cells and therefore for their keeping of viability and capability of subsequent multiplying.

It will be appreciated that this method is in any case compatible with the already known operating methodologies and that a different degree of preparation by the staff putting it into practice is not required.

Finally, the present invention enables low production and sale costs to be achieved both in terms of manufacture of the improvements to isolator devices (even in “aftermarket” installation) and in terms of economy-wise management of the sampling and/or characterization and/or storage and/or analysis works that are nowadays required.

1. An isolator apparatus for collection, parametrical characterization and long-term preservation of organic fluids and/or materials containing stem cells, the isolator comprising:
a work chamber (2) isolated from the external environment;
gloves jutting out at an inside of said work chamber (2), said gloves being connected and sealed with one of the work chamber (2) walls, an operator being able to accede to said work chamber (2) by use of said gloves;
an inlet chamber (4) for introduction of a sample of fluids and/or materials containing stem cells to be processed, said inlet chamber (4) being connected to the work chamber (2) and being provided with interlocking doors that do not allow direct communication between the work chamber (2) and the external environment, said work chamber (2) being pressurized to a greater pressure than the inlet chamber (4);
an outlet chamber connected with the work chamber (2) and having a sterile sample-collecting bag, said outlet chamber being brought into communication with the work chamber (2) by means of interlocking doors;
sterilizing means acting at least on said sample and/or on said inside of the work chamber (2), said sterilizing means being usable before a single working cycle of the isolator and/or between working cycles relating to samples introduced in the isolator one at a time;
filtering means acting on an atmosphere of said inside of the work chamber (2) and/or an inside of the inlet chamber (4) and/or an inside of the outlet chamber;
cooling means for bringing and keeping said sample at a constant working temperature throughout the inlet chamber (4), the work chamber (2) and the outlet chamber; and
visual imaging means (5) focused on the sample at least within the work chamber (2) and defining a visual path which maintains a complete mechanical and pneumatic/hydraulic isolation of said inside of the work chamber (2) with respect to the external environment.

2. An isolator as claimed in claim 1, wherein said visual imaging means (5) comprise:
a camera capable of image and/or video caption focused on the sample within the work chamber (2);
an optically transparent window integrally inserted and flush-formed in a bottom wall of the work chamber (2) and allowing said visual path to be defined upwards from said camera and said sample; and
hermetic seals interposed between said optically transparent window and a locating cavity realized in said bottom wall of the work chamber (2) wherein said optically transparent window is mounted.

3. An isolator as claimed in claim 2, wherein said camera is located outside the work chamber (2) and has a digital resolution ranging from 1 megapixel to 20 mega pixels and a zoom factor ranging from 10× to 30×.

4. An isolator as claimed in claim 2, wherein it further comprises an attachment hub located adjacent to said optically transparent window along said visual path, said attachment hub bearing said camera and comprising illuminating means aligned with said visual path and conveying a flow of light towards the sample.

5. An isolator as claimed in claim 4, wherein said illuminating means comprise a crown of LEDs surrounding a lens of said camera in proximity of said attachment hub, said attachment hub having a diameter ranging from 1 cm to 3 cm.

6. An isolator as claimed in claim 2, wherein said visual imaging means (5) further comprise optical devices aligned with said visual path to enhance and/or deflect and/or concentrate light and/or visual signals travelling along the visual path itself, said optical devices comprising mirrors, lenses, prisms and devices for deflecting/reflecting/refracting light.

7. An isolator as claimed in claim 1, wherein it further comprises electronic visualization, elaboration, correlation and memory storage means connected at least to said visual imaging means (5) and performing visual image processing, storing and data correlating between a set of static images and/or videos taken on said sample and identification parameters associable to the sample itself, said identification parameters comprising:
a particle count related to one or more chemical compounds within the sample;
a microbiological count related to one or more kinds of cells or organic entities, including stem cells, within the sample; and
information regarding a time of sampling, a provenance of sampling, including an identification of a patient/donor, and environmental conditions of sampling.

8. An isolator as claimed in claim 1, wherein said sterilizing means are capable for introducing a sterilizing agent into the isolator, said sterilizing means acting on the work chamber (2) and/or on the inlet chamber (4) and/or on the outlet chamber.

9. An isolator as claimed in claim 8, wherein said sterilizing means are capable of introducing hydrogen peroxide (H₂O₂) at a temperature ranging from 90° C. to 110° C. over a period of time ranging from 30 seconds to 7 minutes.

10. An isolator as claimed in claim 1, wherein said cooling means are capable of bringing and keeping said sample at a working temperature ranging from 2.5° C. to 5.5° C. within a transitory cooling period ranging from 5 minutes to 15 minutes, said cooling means being capable of keeping said sample within said working temperature for a period of time ranging from 40 minutes to 90 minutes throughout the inlet chamber (4), the work chamber (2) and the outlet chamber.

11. An isolator as claimed in claim 1, wherein it further comprises spraying and/or mixing means active on the sample for adding to it a preserving substance.

12. An isolator as claimed in claim 11, wherein said spraying and/or mixing means are capable of adding a predetermined amount of dimethyl sulfoxide (DMSO) to the sample, said predetermined amount ranging from 9% to 11%, preferably equal to 10%, with respect to a total volume of said sample, said sample being added with dimethyl sulfoxide (DMSO) at a constant working temperature ranging from 2.5° C. to 5.5° C.

13. An isolator as claimed in claim 1, wherein it further comprises positioning means for placing and/or moving said sample at least between a waiting position on said bottom wall of the work chamber (2) and an analysis-enabling position on said bottom wall of the work chamber (2), said analysis-enabling position being aligned with said visual path of the visual imaging means (5).

14. An isolator as claimed in claim 2, wherein it further comprises positioning means for placing and/or moving said sample at least between a waiting position on said bottom wall of the work chamber (2) and an analysis-enabling position on said bottom wall of the work chamber (2), said analysis-enabling position being aligned with said visual path of the visual imaging means (5), and wherein said positioning means comprise:
a sample support having a see-through bottom portion resting inside said visual path in the analysis-enabling position;
an engagement projection connected to the sample support and protruding towards said bottom wall of the work chamber (2), said engagement projection resting outside said visual path in the analysis-enabling position of said sample support; and
a predetermined number of engagement seats located in said bottom wall of the work chamber (2) and counter-shaped to said engagement projection, at least one of said engagement seats bearing said optically transparent window.

15. An isolator as claimed in claim 14, wherein said positioning means further comprise releasable clamp locks to be selectively operated on the sample when it is placed in the sample support.

16. An isolator as claimed in claim 11, wherein it further comprises stirring means active on said sample and/or on said preserving substance within the work chamber (2), said stirring means being connected to a mechanical energy source located outside the work chamber (2) through contactless transmission means.

17. An isolator as claimed in claim 16, wherein said stirring means comprise a stirring body made of ferromagnetic material, said contactless transmission means comprising a spatially rotatable/variable magnetic field acting on said stirring body through walls of said work chamber (2).

18. An isolator as claimed in claim 1, wherein said filtering means are of the “hepa” type and/or are made of gore-tex, said filtering means being active on an admission line of said sterilizing means.

19. An isolator as claimed in claim 1, wherein said outlet chamber comprises selecting means for allowing a separate extraction of said sample and of waste materials deriving from operations performed on the sample inside the work chamber (2)s.

20. An isolator as claimed in claim 19, wherein said selecting means comprise a pass-box defining a dedicated waste material exit pathway through a bottom wall of the outlet chamber.

21. A method of collection, parametrical characterization and long-term preservation of organic fluids and/or materials containing stem cells, said fluids and/or materials comprising an amniotic liquid and/or chorionic villi and/or placenta, the method being performed using an isolator according to claim 1 and comprising the following steps:
providing a sample of organic fluid and/or material containing stem cells that is closed and/or separated from the external environment, said sample having a total volume ranging from 1.8 cm³ to 10 cm³;
bringing the sample to a constant working temperature ranging from 2.5° C. to 5.5° C. within a transitory cooling period ranging from 5 minutes to 15 minutes;
adding a predetermined amount of a preserving substance ranging from 9% to 11% with respect to said total volume of said sample, said sample being kept at said constant working temperature; and
keeping said sample added with said preserving substance at said constant working temperature for a period of time ranging from 40 min to 60 min throughout an inlet chamber (4), a work chamber (2) and an outlet chamber of said isolator.

22. A method as claimed in claim 21, wherein it further comprises the following steps:
detecting a set of static images and/or videos taken on said sample;
identificating parameters associable to the sample itself, said identification parameters comprising:
a particle count related to one or more chemical compounds within the sample;
a microbiological count related to one or more kinds of cells or organic entities, including stem cells, within the sample; and
information regarding a time of sampling, a provenance of sampling, including an identification of a patient/donor, and environmental conditions of sampling; and
correlating and storing said set of static images and/or videos and said identificating parameters to said sample.

23. A method as claimed in claim 21, wherein it further comprises the following steps:
freezing the sample;
transferring the sample from the isolator into cryogenic storage means; and
allocating data related to said transferring step of said sample into cryogenic storage means with said set of static images and/or videos taken on said sample and with said identificating parameters associable to the sample itself.

24. A method as claimed in claim 23, wherein said step of freezing the sample is carried out on non-hematic samples and on previously taken samples free of meconium.

25. A method as claimed in claim 21, wherein said step of providing a sample is performed through amniocentesis and/or a chorionic villus sampling procedure (CVS) and/or through selection of a predetermined amount of placenta.
